(19) United States
(12) Patent Application Publication
(10) Pub. No.: US 2005/0267417 A1
(43) Pub. Date: Dec. 1, 2005

(54) Irrigating biopsy inlet valve

(76) Inventors: Dean J. Secrest, Concord, OH (US); Marlin E. Younker, Lantana, FL (US); Christopher J. Kaye, Concord, OH (US)

Correspondence Address: CALFEE HALTER & GRISWOLD, LLP, 800 SUPERIOR AVENUE, SUITE 1400, CLEVELAND, OH, 44114, US

(21) Appl. No.: 11/137,636

(22) Filed: May 25, 2005

Related U.S. Application Data
(60) Provisional application No. 60/574,071, filed on May 25, 2004.

(51) Int. Cl. A61M 5/00

(52) U.S. Cl. 604/247; 604/246; 600/101; 604/267

(57) ABSTRACT

A valve for use in irrigating, accessing and sealing the instrument channel of an endoscope is disclosed. The valve includes a body having a first end, a second end, and an outer circumferential surface. The second end is adapted for connection to an instrument channel inlet port. The outer circumferential surface may define a side entry port. The valve includes a flip cap having an aperture therethrough. The cap is removably insertable into the first end for providing sealable access to the internal passage. The valve may include a tube having an inlet end, an outlet end, and a check valve disposed at the inlet end. The outlet end is fixed to the side entry port. A fluid may be dispensed through the check valve to irrigate the instrument channel. The valve may include a seal curtain member for providing sealable access to the instrument channel.

### CROSS-REFERENCE TO RELATED APPLICATIONS

This non-provisional application claims the benefit of U.S. Provisional Patent Application No. 60/574,071, entitled “Irrigation Biopsy Inlet Valve,” filed May 25, 2004, which is hereby incorporated in its entirety.

### FIELD OF THE INVENTION

The present invention relates to valves and more particularly to an irrigating biopsy inlet valve for use with an endoscope.

### BACKGROUND OF THE INVENTION

Endoscopes are well-known in the art and are commonly used for numerous medical procedures that involve intubation of the esophagus and gastro-intestinal tract. As such, many devices and accessory instruments have been developed that relate to the operation of an endoscope. One such device well-known in the endoscopic art field is commonly referred to as a biopsy channel inlet valve, or BCIV.

A BCIV is used to provide sealable access to the accessory instrument channel of an endoscope. In the beginning of a procedure, the body cavity or lumen is insufflated to provide a lumen for visualization and insertion of an accessory instrument. In doing so, a positive pressure is created in the body and in the channel. The pressure is then retained or held back by use of a BCIV. Further, the valve is required to seal off the instrument passing through the channel. At the end of the procedure, the instrument is removed. A biopsy valve is designed with internal ridges to squeegee blood and other body fluids from the outer surface of an instrument as it is removed from the channel.

During use of these instruments, a need typically arises to irrigate the internal work site and sometimes, also the instrument channel itself. A physician's view of the internal work site can be clouded by various body fluids or debris. A clear view is important for a successful procedure. Conventionally, doctors have removed the accessory instrument, inserted a blunt instrument such as a syringe through a valve sealing an inlet port to the channel and merely sprayed water down the channel. This method has created several problems, such as for example, the valve becomes damaged, the accessory instrument must be removed during irrigation to prevent damage, and undesired time is added to the overall length of the endoscopic procedure. Further, existing valves require manual cleaning between use for disinfection.

The present invention is an improved valve for providing irrigation of the instrument channel of an endoscope. The valve provides sealable access to an inlet port of the instrument channel, is not susceptible to damage during endoscopic procedures, has increased durability over prior art designs, does not require the removal of the accessory instrument during irrigation, and reduces the time of the overall endoscope procedure by permitting remote irrigation access by a doctor or assisting nurse.

The device is made of a relatively inexpensive flexible plastic. Consequently, the device is effectively disposable after one use which eliminates the need to manually clean, flush, and dry existing valves, the threat of cross-contamination from stored valves, and the requirement for disinfection in general. Further, the risk of health worker exposure to infectious body fluid is reduced by eliminating the use of reprocessed valves that are in poor condition and susceptible to leaking

### SUMMARY OF THE INVENTION

In an illustrated embodiment of the invention, a valve for providing resealable access to the instrument channel of an endoscope is disclosed.

In an embodiment, a valve includes a body and a flip cap. The body includes a first end, a second end, and an outer circumferential surface. The body defines an internal passage leading from the first end to the second end, wherein the second end is adapted for connection to an endoscope instrument channel inlet port. The flip cap has a center aperture therethrough and is secured to the body in a spaced relation from the first end.

The flip cap is removably connectable to the first end for providing sealable access to the internal passage. A fluid may be dispensed through the internal passage to irrigate the instrument channel. In another embodiment, a valve apparatus includes a body having a side entry port, flip cap and a tube. The tube has an inlet end and an outlet end. The outlet end is secured to the side entry port. A fluid may be dispensed through the inlet end to irrigate the instrument channel.

In yet another embodiment, a valve apparatus includes a body and a seal curtain. The body has an open end, interior walls defining an inner cavity, and an outer circumferential surface defining a side entry port. The open end is adapted for connection to an endoscope instrument channel inlet port. The seal curtain member has a top section and a flexible curtain portion. The curtain member is removably insertable into the inner cavity for providing sealable access to the instrument channel from the side entry port. Further features and advantages of the invention will become apparent from the following detailed description made with reference to the accompanying drawings.

The Detailed Description of the Invention merely describes preferred embodiments of the invention and is not intended to limit the scope of the claims in any way. Indeed, the invention as described by the claims is broader than and unlimited by the preferred embodiments, and the terms in the claims have their full ordinary meaning.

### BRIEF DESCRIPTION OF THE DRAWINGS

FIG. 1a is a perspective view of an irrigation biopsy inlet valve constructed in accordance with an embodiment of the present invention, showing the valve in use with an endoscopic instrument;

FIG. 1b is a perspective view of the valve illustrated in FIG. 1a, showing the valve in use during an irrigation procedure;

FIG. 2 is an exploded cross-sectional view of the valve illustrated in FIG. 1a, showing an adapter inserted axially through a top portion of the valve;

FIG. 3a is a cross-sectional view of the valve illustrated in FIG. 2, showing a cap in a closed position;

FIG. 3b is an exploded cross-sectional view of the valve illustrated in FIG. 2, showing a cap in a closed position and a flap member under pressure;

FIG. 4 is a cross-sectional view of a portion of the valve illustrated in FIG. 2, shown along the line 4-4 as designated in FIG. 3a;

FIG. 5 is a perspective view of a valve constructed in accordance with another embodiment of the present invention, showing the valve in use during an irrigation procedure;

FIG. 6 is an exploded cross-sectional view of the valve illustrated in FIG. 5, showing a cap in a closed position;

FIG. 7 is a perspective assembly view of a valve constructed in accordance with yet another embodiment of the present invention;

FIG. 8 is a cross-sectional view of the valve illustrated in FIG. 7, showing the valve assembled;

FIG. 9 is a cross-sectional view of the valve illustrated in FIG. 7, showing the valve assembled and an instrument inserted into the endoscope channel;

FIG. 10 is a cross-sectional view of another embodiment of the present invention, showing a valve with a side seal and a large instrument aperture seal;

FIG. 11 is a cross-sectional view of another embodiment of the present invention, showing a valve with an integral check valve and a detachable inlet line; and

FIG. 12 is a side view of a purpose built adapter.

### DESCRIPTION OF THE INVENTION

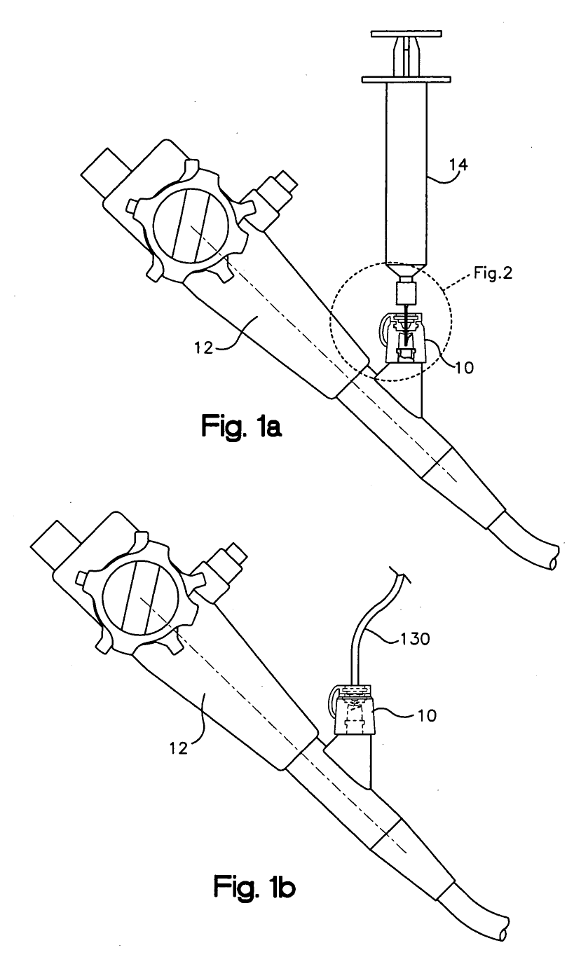

Referring now to the drawings, FIG. 1a is a perspective view of an irrigation biopsy inlet valve 10 constructed in accordance with an embodiment of the present invention. The valve 10 is used to provide sealable access to an instrument channel of an endoscope 12. The valve is highly adaptable for use in a stored or capped position, to allow access by an instrument, or for irrigation of the endoscope instrument channel. An endoscopic instrument 130 is shown inserted through the valve 10. It should be understood by those with ordinary skill in the art that a wide variety of endoscopic instruments can be used in the practice of the present invention, such as for example, snares, retrieval nets or forceps.

The valve 10 is designed to connect to an instrument inlet port of the endoscope 12. FIG. 1b is a perspective view of the valve illustrated in FIG. 1a, showing the valve in use during an irrigation procedure. A common 60 cc syringe 14 or the like can be used by an operator to clean the instrument channel of the endoscope 12. It should be understood by those with ordinary skill in the art that a wide variety of irrigation sources can be used in the practice of the present invention, such as for example, physician operated foot pumps.

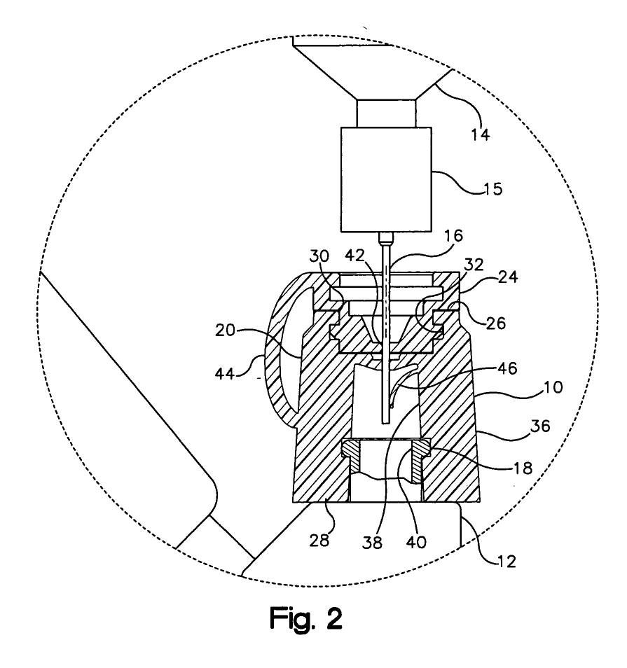

FIG. 2 is an exploded cross-sectional view of the valve 10 illustrated in FIG. 1a, showing a cannula 16 inserted axially through a top portion of the valve 10. The valve includes a body 20 and a flip cap 24. As shown, the body 20 and the flip cap 24 are integrally molded as one piece of a flexible plastic material. It should be understood by others with ordinary skill in the art that the body 20 and flap cap 24 can be constructed from any suitable material. Further, the body 20 and flap cap 24 can be separate pieces.

The valve 10 is press fit over the endoscope instrument channel inlet port 18 and remains in place during normal use.

The body 20 is generally thimble shaped and includes a first end 26 and a second end 28. The body 20 has outer circumferential surface 36 extending between the two ends 26, 28. The first end 26 is proximal to the health worker and defines an opening 30 for insertion of an endoscopic instrument. The opening 30 leads to an interior cavity defined by an irregular surface 32. This surface acts to squeegee blood and body fluid from the instrument during removal. As discussed, the second end 28 is adapted for connection to an endoscope instrument channel inlet port 18.

The body 20 defines an internal passage 38 leading from the first end 26 to the second end 28. This passage provides access to the instrument channel 40 of the endoscope 12. As shown, the passage 38 can be used to transport fluid by a syringe to the instrument channel 40. Further, the passage 38 can be used to provide access to the instrument channel 40 by an endoscopic instrument.

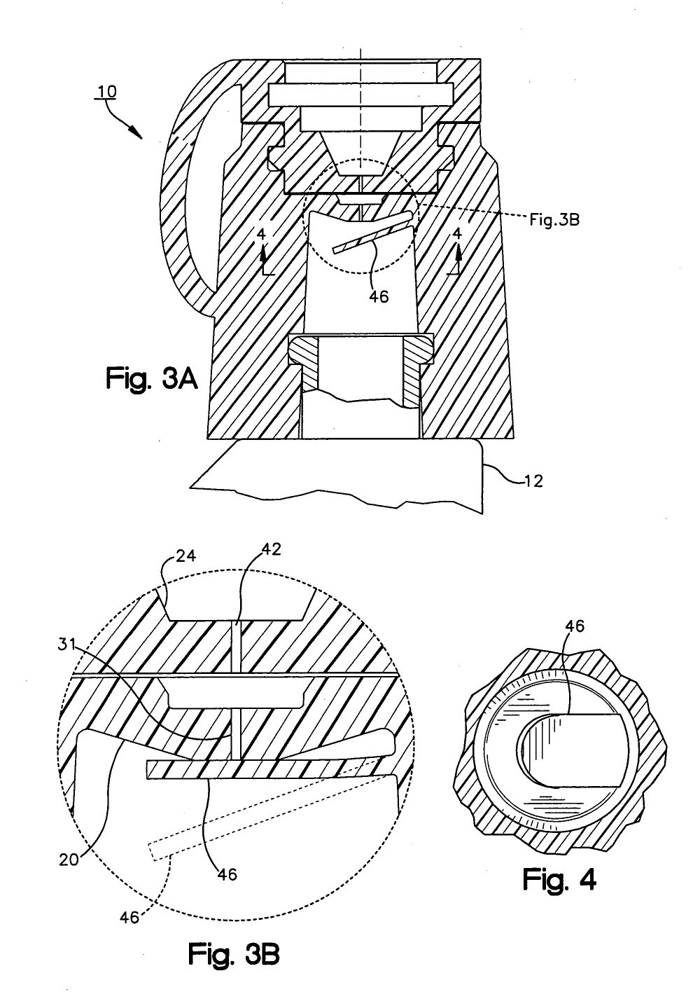

Referring again to FIG. 2, a flip cap 24 is illustrated secured to the body 20 in a closed position. The flip cap has a slit-shaped center aperture 42 therethrough. This slit is co-axial with a slit 31 in the body 20. When in an open position, the flip cap 24 is secured to the body 20 in a spaced relation from the first end 26 by a tether 44. As shown, the tether 44, flip cap 24, and body 20 are one integral piece. In the installed position shown in FIG. 2, the flip cap 24 provides sealable access to the internal passage 38. FIG. 3a is a cross-sectional view of the valve illustrated in FIG. 2.

The valve 10 further includes a flap member 46 internally mounted to the body 20 on an interior wall of the internal passage 38. The flap member 46 is open in relaxed conditions and hangs in a slight downward direction as best seen in FIG. 3A. However, when pressure from fluids in the instrument channel 40 is in a direction toward the first end 26, the flap member 46 inhibits fluid movement in that direction. FIG. 3b shows that flip cap 24 in a closed position and the flap member 46 under pressure. FIG. 4 is an cross-sectional view of the valve 10 along the line 4-4 as designated in FIG. 3a.

Consequently, a bottom view of the flap member 46 is shown. The flap member 46 is shown as one integral piece with the tether 44, flip cap 24, body 20 and constructed of flexible plastic.

A fluid may be dispensed through the internal passage 38 in a direction from the first end 26 to the second end 28 to irrigate the instrument channel 40. The embodiment shown includes a purpose built adapter 15. The adapter includes a specialized sized cannula 16 adapted for insertion through the center aperture slit 42 of the flip cap 24. The cannula is used to engage with the cap 24 in a way that minimally distends the cap slit 42.

In other words, the cannula is only large enough to pierce the slit 42 but not large enough to open the slit sufficiently to allow fluids to travel through the slit in a direction away from the instrument channel. This also advantageously maintains the integrity of the slit during the procedure.

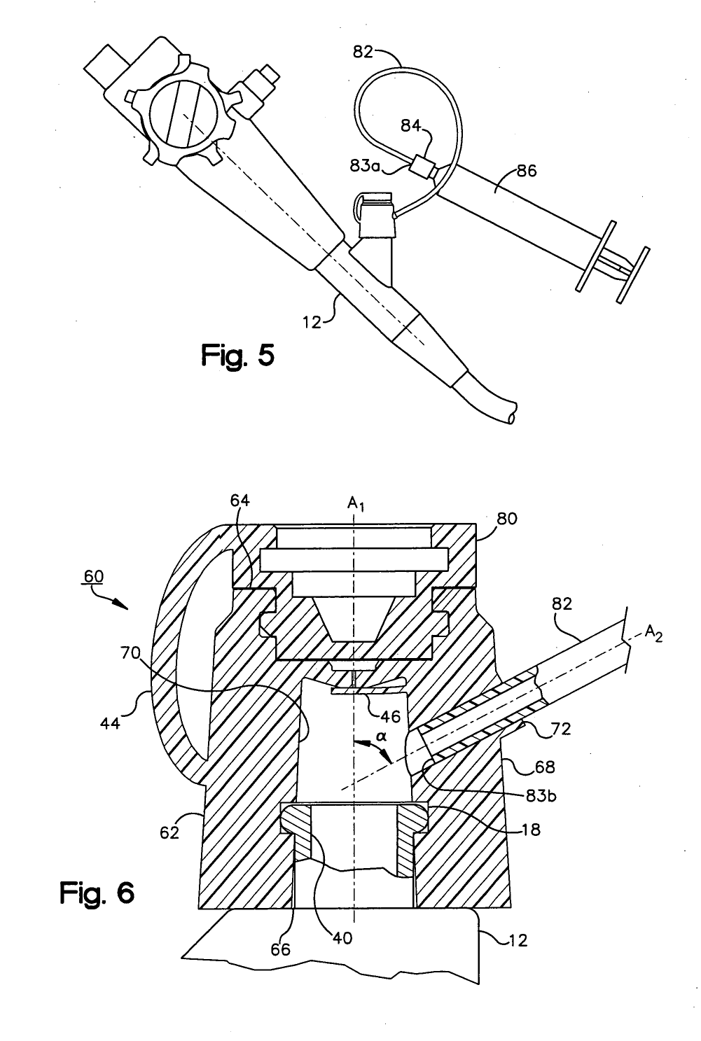

Referring now to FIGS. 5 and 6, a perspective assembly view of a valve apparatus 60 constructed in accordance with yet another embodiment of the present invention is shown. The apparatus is shown installed onto the instrument port 18 of an endoscope 12.

The apparatus 60 includes a remote irrigation feature that allows a nurse or other technician to perform an irrigation procedure remotely from the endoscope inlet port.

Consequently, an intra-procedural irrigation can occur without interruption of the physician. FIG. 6 is an exploded cross-sectional view of the valve 60 illustrated in FIG. 5, showing a cap in a closed position.

The apparatus includes a body 62, a flip cap 80 and a tube 82. The body 62 has a first end 64, a second end 66, and an outer circumferential surface 68. The body 62 defines an internal passage 70 leading from the first end 64 to the second end 66. The second end 66 is adapted for connection to an endoscope instrument channel inlet port 18. The outer circumferential surface 68 defines a side entry port 72.

The flip cap 80 is shown in FIG. 6 in a closed position. The flip cap 80 is secured in a spaced relation from the body 62 by a tether 44. The flip cap 80 is removably connectable to the first end 64 for providing access to the internal passage 70. The flip cap may have a center aperture therethrough (not shown).

The tube 82 can be used by a technician to irrigate the instrument channel 40. The length of the tube from the body 62 allows a technician to perform the irrigate function without disruption to the physician. The tube 82 has an inlet end 83a and an outlet end 83b. The outlet end 83b is secured to the side entry port 72 by any suitable method known in the art.

The port 72 and outlet end 83b of the tube define a longitudinal axis A₂ which is oriented at an angle α with respect to A₁. Fluid may be dispensed through the outlet end 83b in a direction toward the instrument channel 40. A syringe 86 may be connected to a check valve 84 to irrigate the instrument channel 40. It is believe that an angle α of less than 90 degrees promotes flow down the instrument channel 40 and generally improves irrigation. Other irrigation apparatus may be connected to the check valve in the practice of the present invention. As illustrated in FIG. 5, the inlet tube 82 includes a check valve 84 at the inlet end 83a. The check valve 84 allows for a physician, nurse or other technician to control the flow of irrigation fluid to the instrument channel 40 remotely from the inlet port 72, advantageously maintaining the port seal. This feature further increases the durability of the apparatus by eliminating damage from cannula insertion through the valve body 62 and valve cap 80.

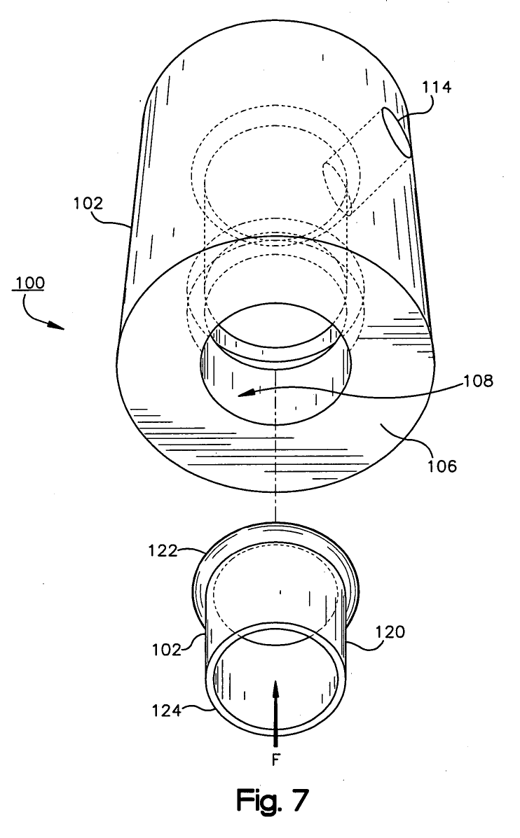

The apparatus includes a flap member 46 that inhibits fluid movement in a direction from the instrument channel 40 to the first end 64 of the body 62. As shown, the tether 44, flip cap 80, body 62 and flap member 46 are one integral piece constructed of flexible plastic. Referring now to FIG. 7, a perspective assembly view of a valve apparatus 100 constructed in accordance with yet another embodiment of the present invention is shown.

The valve 100 is a two piece assembly and includes a main body 102 and a seal curtain 104. The pieces 102, 104 are each constructed of a flexible plastic. Prior to use with an endoscope, an operator applies a force in the direction F to press fit the seal curtain 102 into the main body 104.

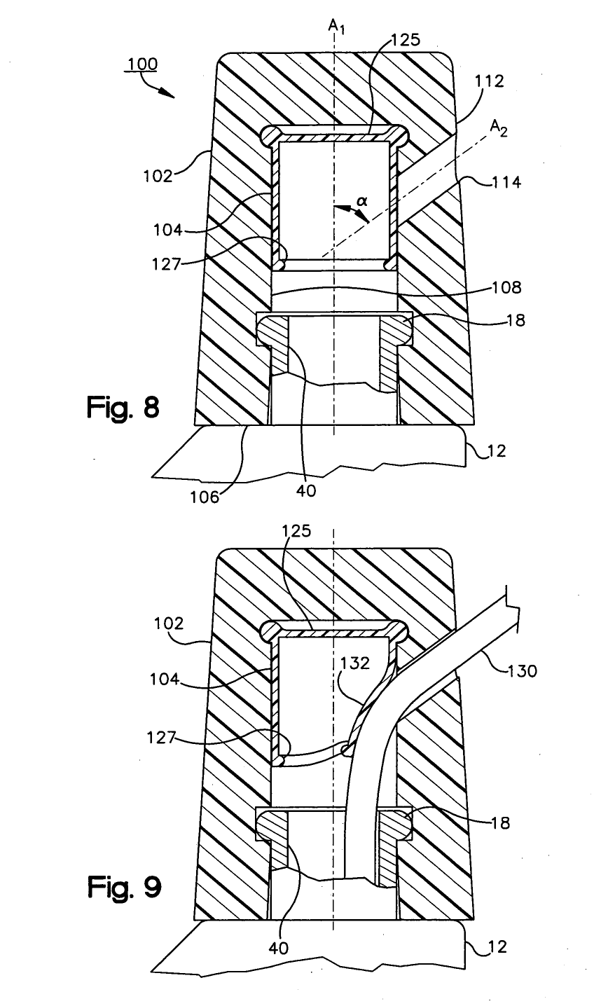

A cross-sectional view of the valve assembly 100 is illustrated in FIG. 8 in an assembled position. The body 102 has an open end 106 that is adapted for connection to an endoscope instrument channel inlet port 18. The body also include interior walls that define an inner cavity 108. As shown, the inner cavity is uniformly shaped about a center longitudinal axis A₁ of the body 102. The body may be constructed of any suitable material, such as for example, a flexible molded plastic.

The body provides access to the instrument channel 40 of the endoscope 12. An outer circumferential surface 112 of the body 102 defines a side entry port 114. To be discussed later in greater detail, the side entry port 114 is adequately sized for insertion of a common endoscope instrument 40, such as for example, a snare, a retrieval net, or forceps. The port 114 defines a longitudinal axis A₂ which is oriented at an angle α with respect to A₁. Fluid may be dispensed through the side entry port 114 in a direction toward the instrument channel 40. An operator may use any conventional device, such as for example, a needle and syringe, to irrigate the instrument channel 40. It is believe that an angle α of less than 90 degrees promotes flow down the instrument channel 40 and generally improves irrigation.

The seal curtain member 104 has a flexible cylinder-shaped portion 120. The cylinder-shaped portion 120 has a top edge 122 and a bottom edge 124. As discussed, the curtain member 104 is removably insertable into the inner cavity 108 with the top edge 104 leading. As shown, the seal curtain member 104 includes a disk-shaped top section 125 joined to the top edge 122. The curtain member is closed in relaxed conditions and seals the inlet port 114. Flow back pressure from the instrument channel 40 actually tightens the seal, forcing the curtain against the interior wall of the valve body and preventing fluid escape. To aid strength and rigidity to the seal curtain member 104, a stabilizing ring 127 is molded on the inside of the bottom edge 124. It is believed that this ring improves seal integrity with the body inner cavity wall.

In the assembled position, the valve 100 provides sealable access to the instrument channel 40 of an endoscope 12. Referring now to FIG. 9, an exploded cross-sectional view of the valve 100 is shown with an instrument 130 inserted into the instrument channel 40. A section 132 of the seal curtain 104 is pressed away from the body 102 by the instrument 130. After removal of the instrument, the section 132 will return to a contiguous position with the cavity 108 inner wall. It should be understood by those with ordinary skill in the art that a wide variety of endoscopic instruments could be used in the practice of the present invention.

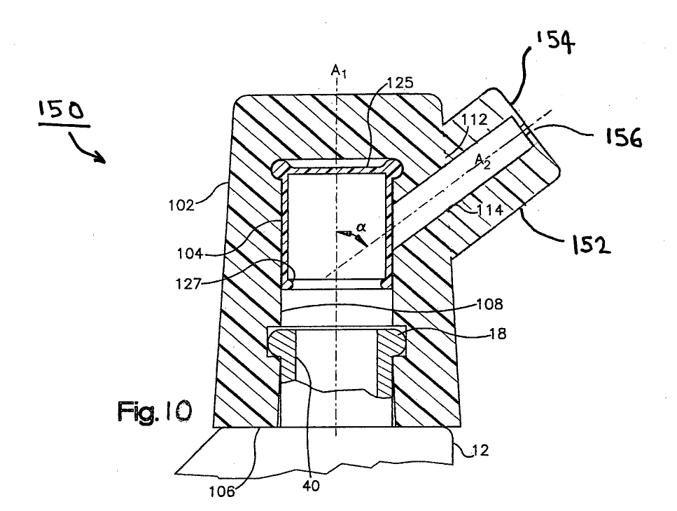

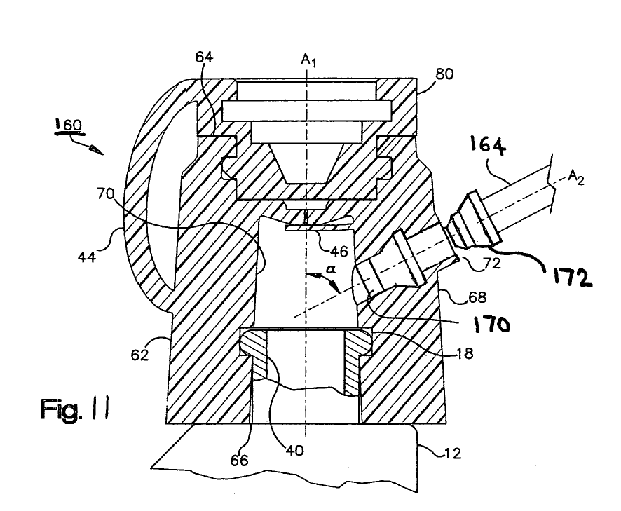

Referring now to FIG. 10, a cross-sectional view of another embodiment of the present invention is shown. The device 150 include a side port 152 suitable for use with larger diameter endoscope instruments. The face 154 of the port defines an inlet 156 through which an instrument can be inserted. The inlet 156 can be for example a slit, a circular-shaped aperture, or a combination of the two. The inlet port seal combines with the seal curtain 104 to seal large instruments.

FIG. 11 is a cross-sectional view of yet another embodiment of the present invention. A device 160 includes a side port 72 that defines the opening of an internal passage leading to an integral valve 170, such as for example, a check valve or a duck bill valve. The inlet port 72 is sized for insertion of a detectable inlet line including a press fit connector 172 and a tube 164. The check valve 170 can be used to control flow in the proximal direction toward the instrument channel 40 and prohibit flow in the opposite direction.

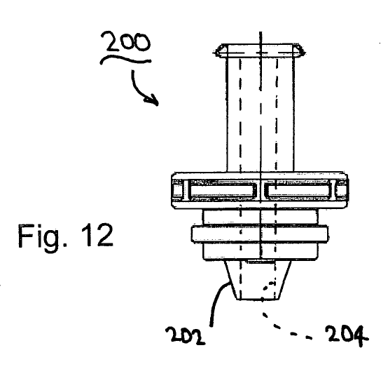

A purpose build adapter 200 for use with a valve of the present invention is shown in FIG. 12. The adapter 200 includes a polygon shape 202 protrusion at its distal end. The protrusion is specialized sized for matting to the first end of the body 20 wit the flip cap removed. The insertion effectively opens the center aperture slit 31 of the valve body 20. The adapter is used to engage with the body 20 in a way that minimally distends the slit 31. In other words, the adapter is only large enough to pierce the slit 31 but not large enough to open the slit sufficiently to allow fluids to travel through the slit in a direction away from the instrument channel. This also advantageously maintains the integrity of the slit during the procedure.

While several embodiments of the invention has been illustrated and described, the present invention is not to be considered limited to the precise constructions disclosed. Various adaptations, modifications and uses of the invention may occur to those skilled in the arts to which the invention relates. It is the intention to cover all such adaptations, modifications and uses falling within the scope or spirit of the annexed claims.

1. An irrigating biopsy inlet valve for providing sealable access to an instrument channel of an endoscope, the valve comprising:
a. a body having a first end, a second end, and an outer circumferential surface and defining an internal passage leading from said first end to said second end, wherein said second end is adapted for connection to an endoscope instrument channel inlet port;
b. a cap having a center aperture therethrough and removably connectable to said first end for providing sealable access to said internal passage; and
c. a flap member internally mounted to said body in said internal passage, wherein said flap member inhibits fluid movement in a direction from said instrument channel to said first end of said body;
d. wherein a fluid may be dispensed through said internal passage in a direction from said first end to said second end to irrigate said instrument channel.

2. The valve of claim 1 further comprising a cannula, said cannula adapted for insertion through said center aperture of said flip cap when said flip cap is inserted into said first end of said body.

3. The valve of claim 1 further comprising a purpose-built adapter for mounting on said body to minimally distend a center aperture in said body first end.

4. The valve of claim 1 wherein said outer circumferential surface defines a side entry port for injecting irrigation fluid to said instrument channel via said internal passage.

5. The valve of claim 1 wherein said body is constructed of a flexible plastic.

6. The valve of claim 1 wherein said outer circumferential surface defines a side entry port, said port defining a downward flow path angled less than 90 degrees from a longitudinal axis of said internal passage.

7. The valve of claim 1 wherein said center aperture is a slit.

8. The valve of claim 1 further comprising an adapter, wherein said outer circumferential surface defines a side entry port and said adapter comprises a duck-bill valve, said duck bill valve insertable into said side entry port such that said adapter provides access to said internal passage open and seals said internal passage when closed.

9. The valve of claim 1 wherein said cap is secured to said body in a spaced relation from said outer circumferential surface by a tether.

10. The valve of claim 1 wherein said body, said cap and said flap member are one integral piece.

11. The valve of claim 1 wherein said valve is constructed of a clear plastic.

12. A valve apparatus for providing sealable access to an instrument channel of an endoscope, the valve comprising:
a. a body having a first end, a second end, and an outer circumferential surface and defining an internal passage leading from said first end to said second end, wherein said second end is adapted for connection to an endoscope instrument channel inlet port and said outer circumferential surface defines a side entry port;
b. a flip cap secured in a spaced relation from said body, said flip cap removably connectable to said first end for providing sealable access to said internal passage; and
c. a tube having an inlet end and an outlet end, said outlet end secured to said side entry port;
d. wherein a fluid may be dispensed through said tube in a direction from said inlet end to said outlet end to irrigate said instrument channel.

13. The apparatus of claim 12 further comprising a check valve disposed at said inlet end, wherein a fluid may be dispensed through said check valve to irrigate said instrument channel.

14. The apparatus of claim 12 wherein said side entry port defines a downward flow path angled less than 90 degrees from a longitudinal axis of said internal passage.

15. The apparatus of claim 12 wherein said body is constructed of a flexible plastic.

16. The apparatus of claim 12 wherein said body and said flip cap are one integral piece.

17. The apparatus of claim 12 further comprising a flap member internally mounted to said body in said internal passage, wherein said flap member inhibits fluid movement in a direction from said instrument channel to said first end of said body.

18. The apparatus of claim 12 wherein a flip cap has a center aperture therethrough.

19. The valve of claim 12 wherein said valve is constructed of a clear plastic.

20. The valve of claim 12 further comprising a purpose-built adapter for mounting on said body to minimally distend a center aperture in said body first end.

21. A valve apparatus for providing sealable access to an instrument channel of an endoscope, the apparatus comprising:
a. a body having an open end, interior walls defining an inner cavity, and an outer circumferential surface defining a side entry port, wherein said open end is adapted for connection to an endoscope instrument channel inlet port; and
b. a seal curtain member comprising a flexible cylinder-shaped portion having a top edge and a bottom edge, said curtain member being removably insertable into said inner cavity said top edge leading for providing sealable access to said instrument channel from said side entry port.

22. The apparatus of claim 21 wherein a fluid may be dispensed through said side entry port to irrigate said instrument channel.

23. The apparatus of claim 21 wherein said side entry port defines a downward flow path angled less than 90 degrees from a longitudinal axis of said body.

24. The apparatus of claim 21 wherein said body is constructed of a flexible plastic.

25. The apparatus of claim 21 further comprising an integral valve for control of fluid flow through said side entry port.

26. The apparatus of claim 21 further comprising a detachable inlet tube sized for insertion into said side entry port.

27. The apparatus of claim 21 wherein said side entry port is sized for insertion of an endoscope instrument.

28. The apparatus of claim 21 wherein a fluid may be dispensed through said side entry port in a direction toward said endoscope instrument channel inlet port to irrigate said instrument channel.

29. The apparatus of claim 21 wherein said seal curtain member comprises a disk-shaped top section joined to said top edge.

30. The apparatus of claim 21 wherein said seal curtain bottom edge comprises a stabilizing ring.

31. The valve of claim 21 wherein said valve is constructed of a clear plastic.
